(19) United States
(12) Patent Application Publication
(10) Pub. No.: US 2017/0128284 A1
(43) Pub. Date: May 11, 2017

(54) ABSORBENT ARTICLE EXHIBITING CONTROLLED DEFORMATION IN USE

(71) Applicant: SCA Hygiene Products AB, Göteborg (SE)

(72) Inventors: Hanna ESPING ÖSTLIN, Göteborg (SE); Edward GUIDOTTI, Göteborg (SE)

(73) Assignee: SCA Hygiene Products AB, Göteborg (SE)

(21) Appl. No.: 15/318,077

(22) PCT Filed: Jun. 13, 2014
(86) PCT No.: PCT/SE2014/050716

§ 371 Date: Dec. 12, 2016

(51) Int. Cl. A61F 13/536; A61F 13/494; A61F 13/49

(52) U.S. Cl. CPC A61F 13/536; A61F 13/49017; A61F 13/4902; A61F 13/49406; A61F 2013/530875

(57) ABSTRACT

An absorbent, disposable article includes an absorption body having longitudinally extending edges is disposed between the liquid-permeable casing layer and the substantially liquid-impermeable backside layer. The article includes a longitudinally extending centre line, a crotch area, a front end area and a back area. The absorption body includes a first absorption layer facing the user and a second absorption layer facing away from the user during. At least one elastic element is disposed between the liquid-permeable casing layer and the backside layer outside of the respective longitudinally running edge of the absorption body. The elastic elements have a return force between 0.30 N and 0.80 N at an extension of 70% of the accessible extension. The first absorption layer includes a first longitudinal opening disposed in the crotch area and the second absorption layer includes a first longitudinal compression disposed in the crotch area.

### TECHNICAL AREA

The present disclosure relates to an absorbent, disposable article comprising a casing layer permeable to liquid provided to be used facing the user and comprising a substantially liquid-impermeable backside layer provided to be used turned away from the user during use. An absorption body comprising longitudinally running edges is disposed between the liquid-permeable casing layer and the substantially liquid-impermeable backside layer.

The article and the absorption body comprise a longitudinally running centre line, a crotch area, a front end area and a back end area. The absorption body comprises a first absorption layer provided to be turned facing the user during use and a second absorption layer provided to be turned away from the user during use.

The liquid-permeable casing layer and the backside layer extend past the longitudinally running edges of the absorption body. At least one elastic element is disposed between the liquid-permeable casing layer and the backside layer past the respective longitudinally running edge. The elastic elements have a return force between 0.30 N and 0.80 N at an extension of 70% of the accessible extension according to the described test method.

The first absorption layer comprises a first longitudinal opening disposed in the crotch area and the second absorption layer comprises a first longitudinal compression disposed in the crotch area.

### BACKGROUND

It is known that absorption bodies intended to be used in absorbent articles must have a sufficient absorption capacity as well as the ability to be able to rapidly receive outflowing liquid when, for example, an adult incontinent wearer of the article involuntarily empties his entire bladder.

Based on what the absorbent article is intended for, the absorption capacity varies within broad limits, for example, a capacity of 4 g is sufficient for a sanitary pad of a smallest size whereas a capacity of over 1000 g is required for the largest articles intended for adult incontinent persons

To manufacture articles with sufficiently high absorption capacity is not a great problem today when there are gel-forming, highly absorbent particles, so-called super absorbers to be mixed into the absorption bodies of the absorbent articles.

A type of problem with today's very thin and hard compressed articles is to arrange that the articles can receive liquid being excreted and hitting the article at a high flow rate. For example, absorbent articles for adult, incontinent users must be able to receive several hundred millilitres of urine excreted in 10 seconds. If the absorption body of the absorbent article is not able to receive and absorb this large flow, there is a high risk that the urine runs on the surface of the article and out over the edge of it.

A solution of the problem of receiving rapidly excreted bodily liquid is to arrange special material layers between the absorption body and the user that are intended to receive and temporarily store liquid in the expectation that the absorbent body succeeds in absorbing the liquid. Examples of such materials are resilient pads of, for example, polyester fibres.

Another existing solution is that today's thin, wide crotch structures form a cup-shaped structure between the wearer's thighs in which the liquid can be temporarily collected before it enters into the absorption body. The absorption bodies of the articles have been provided with compressions, slots, contracting elastic elements or the like in order to control the shaping of the articles when they are pressed together between the wearer's thighs during use.

It is described in patent application GB 2,296,437 (Hansson, et al., Mölnlycke AB) how the shaping of an absorbent article can be controlled when it is exposed to forces that are substantially perpendicular to the longitudinal direction of the article.

The article comprises an absorbent layer that comprises a through slot and a first material layer over one surface of the absorbent layer and a second material layer over the opposite surface of the absorbent layer.

One of the material layers is connected to the absorbent layer adjacent said slot while the other material layer is not connected adjacent said slot. When the article is exposed to compressive forces directed perpendicular to the slot, the article is forced to be bent/turned in the direction away from the non-connected side of the absorption layer.

The patent document WO 2011/105108 A1 (Mukai et al. Uni-Charm) describes a refined variant of an absorbent body that shapes between the users thighs when the article is used.

The absorbent body in WO 2011/105108 A1 is constituted by 2 layers of which the layer that is disposed away from the user comprises a longitudinal opening centrally disposed in the crotch area along the longitudinal central line. The other absorbent layer, which is oriented closest to the wearer of the article, comprises side openings symmetrically placed between the layer's longitudinal centre line and the respective longitudinal edge.

The document shows that the shaping of the absorbent body with longitudinal openings in both absorption layers means that the absorbent article is shaped like a W in the crotch area when it is pressed together between the thighs of a user.

It has turned out, however, that regardless of whether the article was designed for receiving a simple cup shape, a U-shape or a V shape during use or a more sophisticated cup shape, a W shape, there is the problem that the article does not assume the intended shape when it is placed on a user.

For example, it is not unusual that the article assumes an inverted shape when it is exposed to pressure forces from the thighs of the user, which means, for example, that the U-shape instead becomes an inverted U shape (∩ shape) and the W shape becomes an inverted W shape (M shape).

It has also turned out that articles intended to receive a U shape, a V shape or a W shape when they are deformed transversely between the thighs of the user have too low a spring-back force against the thighs of the user, wherein the article is not capable of effectively sealing against the thighs during use.

Therefore, there is a need for improved absorbent articles that can be shaped with greater security in the desired manner when the article is placed and pressed together between a user's thighs. The need is present regardless of whether it is a question of absorbent articles intended for incontinent children or adults.

Furthermore, there is a need for an article that, in the deformed state according to the above, has an increased spring-back force in the transverse direction against the user's thighs during usage.

### SUMMARY

According to the present disclosure, an absorbent disposable article of the initially cited type has been achieved that substantially removes the problems associated with earlier known absorbent articles.

An absorbent article according to the disclosure is distinguished primarily in that the first longitudinal opening and the first longitudinal compression substantially overlap each other.

The arrangement with an absorbent article comprising an absorption body comprising a first and a second absorption layer, comprising a longitudinal opening in the first absorption layer and a longitudinal compression in the second absorption layer that overlap one another means that the absorbent article is deformed in a substantially more predictable manner when it is pressed together between the user's legs than earlier known articles were.

According to an aspect of the disclosure the first longitudinal opening in the first absorption layer and the first longitudinal compressing in the second absorption layer coincide substantially with the longitudinal centre line.

The centring of the opening and the compression forms a centred V shape when an article comprising the absorption body is compressed between a user's thighs.

According to another aspect of the disclosure the first absorption layer comprises a second longitudinal opening disposed in the crotch area between the longitudinal centre line of the absorption layer and a longitudinal edge, wherein the first longitudinal opening is disposed between the longitudinal centre line and the opposite longitudinal edge.

Furthermore, the second absorption layer comprises a second longitudinal compression disposed in the crotch area between the longitudinal centre line and the one longitudinal edge of the absorption layer, wherein the first longitudinal compression is disposed between the longitudinal centre line and the opposite longitudinal edge.

The second longitudinal opening and the second longitudinal compression substantially overlap each other in the same manner as the first longitudinal opening and the first longitudinal compression substantially overlap one another.

The fact that the overlapping openings and compressions are each disposed on each side of the centre line forms a symmetrical U shape when an article comprising the absorption body is compressed between a user's thighs.

According to a further aspect of the disclosure the first absorption layer comprises a third longitudinal compression disposed in the crotch area and substantially coinciding with the longitudinal centre line and the second absorption layer comprises a third longitudinal opening disposed in the crotch area and substantially coinciding with the longitudinal centre line. The third longitudinal compression and the third longitudinal opening overlap one another.

A symmetrical W shape is formed when an absorbent article comprising the absorption body is compressed between a user's thighs.

According to an aspect of the disclosure the article comprises at least one inner side leakage barrier disposed on each side around the article's longitudinal centre line.

The respective inner side leakage barriers are adjacent to the respective longitudinal edge of the absorption body, at least in the crotch area. Furthermore, the respective inner side leakage barriers have a free edge. The respective inner side leakage barriers comprise at least one tensioned elastic element.

The providing of the inner free edge of the inner side leakage barriers with at least one tensioned elastic element means that the barriers assume an upright configuration when the elastic elements are contracted.

According to an aspect, the disclosure is characterized in that the elastic elements disposed in the respective inner side leakage barriers have a return force between 0.20 N and 0.40 N at an extension of 70% of the accessible extension according to the described test method.

According to an aspect of the disclosure the absorbent article comprises an inlet material disposed on the side of the first absorption layer that is intended to be turned toward the user.

A special inlet material in conformity with the present disclosure receives rapidly incoming liquid and is capable of temporarily storing the liquid until the adjacent absorbent layer is able to receive the liquid.

According to another aspect, the disclosure is characterized in that the inlet material is divided into 2 separate parts. One part is disposed between the longitudinal centre line and one longitudinal edge of the absorption body and the other part is disposed between the longitudinal centre line and the other longitudinal edge of the absorption body. The respective part of the inlet material is at a distance from the longitudinal central line and from the adjacent longitudinal edge.

An inlet material divided into two parts is especially advantageous when the absorbent article is entirely folded together around its longitudinal centre line during use, that is, when the one half of the absorbent article rests against the opposite longitudinal half of the article.

According to an aspect of the disclosure, the first absorption layer and the second absorption layer of the absorbent disposable article have substantially the same basis weight and according to another aspect the respective absorption layers have a basis weight between 50 g/m2 and 1000 g/m2.

According to an aspect of the disclosure the respective absorption layer of the absorbent article has a content of highly absorbent polymer material, so-called SAP, between 0% and 80%.

Highly absorbent polymer material, so-called SAP, is capable of absorbing large amounts of urine in relation to its own weight and can furthermore bind the urine substantially more firmly than capillary absorption. The absorbent article has a reduced tendency to become wet again, for which reason the article is experienced as being extremely dry in spite of the fact that it absorbed much liquid.

According to an aspect of the disclosure one absorption layer of the absorbent article has a greater extension longitudinally than the second absorption layer and according to another aspect the front end area and the back end area of the absorption body have a greater width in the transversal direction than the crotch area.

Another aspect of the disclosure is characterized in that the absorbent disposable article is constituted of a baby diaper and a second aspect of it in that it is constituted by an incontinence diaper provided for adult incontinent users.

### BRIEF DESCRIPTION OF THE FIGURES

FIG. 1 shows a perspective view of an absorption body according to a first embodiment of the invention when the two absorption layers of the absorption body are shown separated from one another for greater clarity.

FIG. 2a shows the absorption body of FIG. 1 in which the two absorption layers of the absorption body are disposed on one another.

FIG. 2b shows the absorption body of FIG. 2 in which a special inlet material divided into two parts is disposed on the absorption body.

FIG. 3a shows an absorbent article intended for absorption of urine and comprising an absorption body in conformity with FIGS. 1 and 2.

FIG. 3b shows how the cross section A-A in FIG. 3a is shaped during use when the article is exposed to lateral forces perpendicular to the longitudinal direction from a user's thighs.

FIG. 3c shows how the cross section A-A in FIG. 3a can be shaped during use when the article is exposed to lateral forces perpendicular to the longitudinal direction from a user's thighs when the space between the user's thighs is small.

FIG. 4a shows a perspective view of an absorption body according to an alternative embodiment of the invention in which the two absorption layers of the absorption body are shown separated from one another for greater clarity.

FIG. 4b shows the absorption body of FIG. 4a when the two absorption layers of the absorption body are disposed on one another.

FIG. 4c shows how the cross section B-B in FIG. 4b is shaped during use when the absorption body in FIG. 4b is disposed in an absorbent article exposed to lateral forces perpendicular to the longitudinal direction from a user's thighs.

FIG. 5 shows a perspective view of an absorption body according to a third embodiment of the invention in which the two absorption layers of the absorption body are shown separated from one another for greater clarity.

FIG. 6 shows the absorption body of FIG. 5 when the two absorption layers of the absorption body are disposed on one another.

FIG. 7 shows how the cross section C-C in FIG. 6 is shaped when the absorption body in FIG. 6 is disposed in an absorbent article exposed to lateral forces perpendicular to the longitudinal direction from a user's thighs.

### DESCRIPTION OF EMBODIMENTS

An embodiment of the invention includes an absorbent article comprising an absorption body constituting a part of the absorbent article. The absorption body comprises at least two absorption layers wherein the one absorption layer is oriented toward the user during use and the other absorption layer is oriented away from the user during use.

At least the absorption layer disposed toward the user comprises at least one longitudinal opening and the absorption layer disposed away from the user comprises at least one longitudinal compression.

The two absorption layers are disposed in such a manner that openings and compressions in the two absorption layers cooperate so that the absorption article comprising the absorption body is shaped in a desired manner when it is placed on a user and pressed together crosswise between his or her thighs.

In one embodiment, an absorption body is built into an absorbent article and assumes a cup shape (V or U-shape) when the article is pressed together crosswise between a user's thighs.

The disclosure also relates to absorbent articles comprising absorption bodies that are shaped in accordance with the above. Absorbent articles denote in this connection a plurality of types of absorbent articles such as, for example, all-in-one diapers, diaper pants, belt diapers or menstruation protection.

So-called all-in-one diapers are characterized in that they comprise fastening flaps with which the front and rear waist parts of the diaper are connected when the diaper is applied around the waist of the user. All-in-one diapers usually comprise elastic areas at the leg cut-outs and in connection with the waist area where at least parts of the waist portion usually have elastic members. The fastening flaps comprise elastic areas in many all-in-one diapers. In addition, the fastening flaps can comprise adhesive surfaces that are connected to surfaces on the opposite end portion to which the adhesive fastens. Fastening systems of hook & loop type is currently customarily in use.

So-called pant diapers are characterized in particular in that they have been folded around a substantially transverse fold line in the crotch part of the pant diaper during manufacture and are subsequently joined together at the waist. These types of diapers are intended to be applied on a user exactly like a pair of briefs that is, drawn over the user's legs. The joining at the waist part of the pant diaper can usually be detached, wherein the pant diaper can be taken off after use without having to be drawn down over the user's feet when it is to be removed. This possibility is especially appreciated when the pant diaper is smeared with faecal matter after use. Pant diapers normally comprise elastic areas in the waist part and around the leg openings.

Pant diapers that can be opened and reclosed also exist. Such pant diapers are supplied folded together at the waist but can be opened, for example, for checking the contents in the article and can then be closed again.

Belt diapers are characterized by that they comprise one, in comparison to the diaper's absorbent part, transverse belt connected either to the front or back transverse edge of the diaper.

When such a belt diaper is applied, the belt is fixed in a first step around the user's waist. The absorbent part of the diaper then hangs loosely from the belt. The absorbent part of the diaper is then led between the user's legs and fastened to the belt, wherein the belt comprises fixing surfaces for clamping fast to fixing members disposed on the absorbent part of the diaper alongside its free transverse edge. The belt and the leg cut-outs are usually elasticised on belt diapers.

Another type of belt diaper has two parts and includes a separate belt and a separate absorbent structure. During use the belt is fastened around the user's waist, after which the absorbent structure is connected to the outside of the belt by hook & loop elements or tape elements at the corners of the absorption structure.

All-in-one diapers, pant diapers or belt diapers can include baby diapers intended for children that are not yet pot-trained or of incontinence protection intended for adult incontinent users.

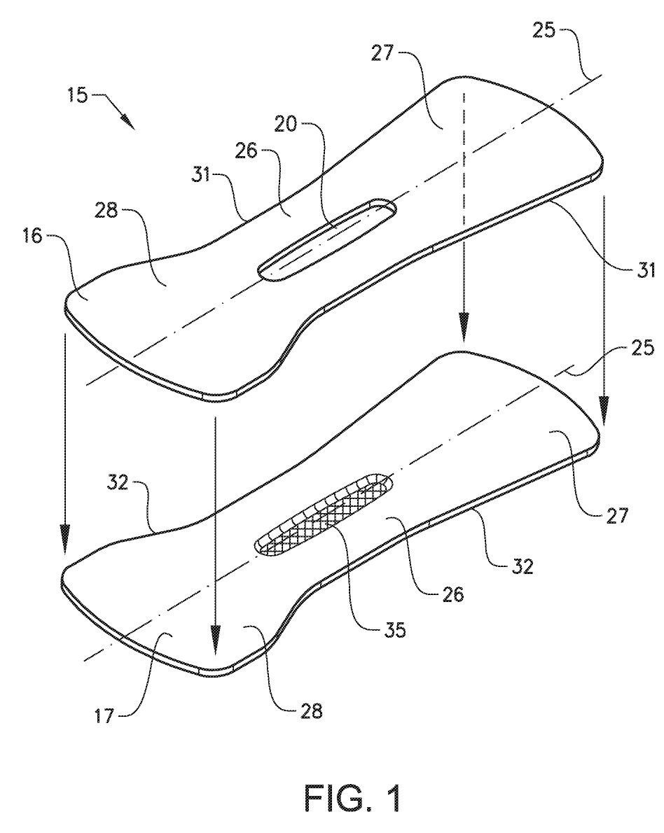

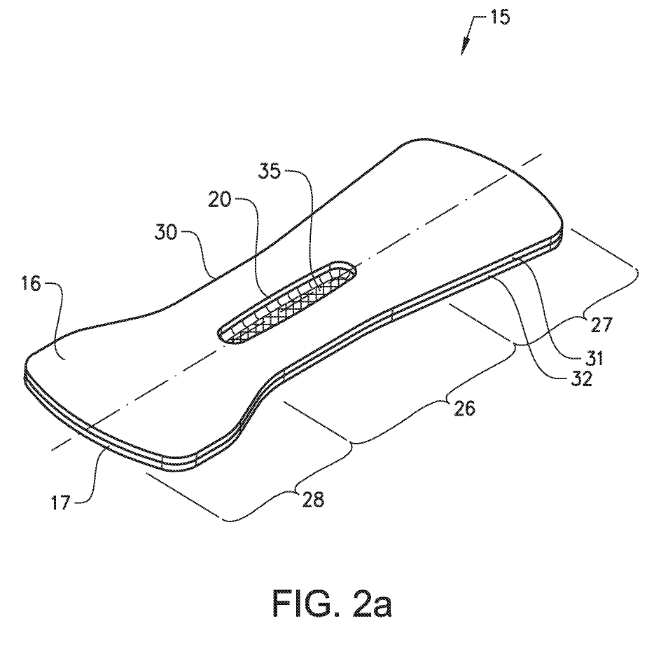

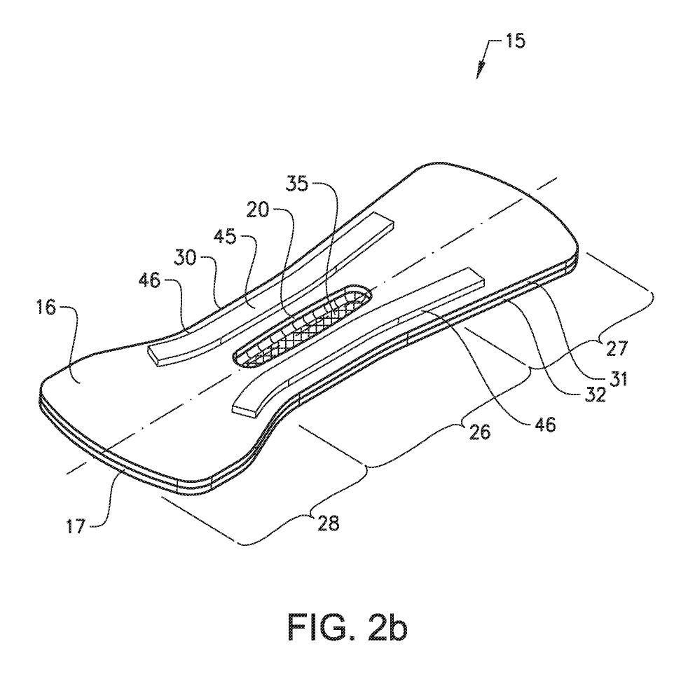

FIGS. 1 and 2 show an absorption body 15 intended to be placed in an absorbent disposable article in conformity with a first aspect of the disclosure. The absorption body 15 comprises a first absorption layer 16 and a second absorption layer 17 comprising, among other things, cellulose fluff pulp.

In FIG. 1 the first and the second absorption layers 16, 17 of the absorption body 15 have been separated from one another in order to increase clarity while FIG. 2a shows the absorption body 15 as it is disposed when it is applied into an absorbent article.

Alternatively, the absorption body 15 can comprise additional absorption layers.

In many absorption bodies the cellulose fluff pulp is mixed with fibre or particles of a highly absorbent polymer material of the type that chemically binds large amounts of liquid during absorption, forming a liquid-containing gel.

The respective absorption layers 16, 17 can have a basis weight from 50 g/m² up to 1000 g/m² and can contain 0-80% highly absorbent polymeric material, so-called SAP.

Absorption body 15 can furthermore comprise components for improving various properties of the absorption body 15. Examples of such components are binding fibres, various types of liquid-spreading layers or fibres, form-stabilising components, reinforcement fibres or the like. Such components are not shown in any of the figures.

The absorbent layers 16, 17 are usually produced in-line in the same machine that manufactures the entire absorbent article; however, it is also possible that the absorption layers 16, 17 are produced by prefabricated absorption material. Prefabricated absorption material is usually supplied in roll form, wherein the material is cut and folded to the prescribed configuration. Prefabricated absorption material can contain the same components as absorption bodies produced in-line. Binding fibres are in principle a necessary component in prefabricated absorption materials so that they stay together during handling.

The absorption body 15 comprises a front end area 27, a back end area 28, a crotch area 26 disposed between the end areas 27, 28, and longitudinal edges 30. The absorption body 15 has an hourglass shape, wherein the crotch area 26 is narrower than the front and the back end areas 27, 28.

Alternatively, the absorption body 15 can have another shape, for example are substantially rectangular absorption bodies common.

The absorption body 15 has a longitudinal centre line 25.

The first absorption layer 16 has longitudinal edges 31.

A longitudinal opening 20 is disposed in the crotch area 26 of the first absorption layer 16 and coincides with the centre line 25. The opening 20 has its main extension in the crotch area 26 but can also extend into both the front and the back end areas 27, 28. Opening 20 extends longitudinally 30% of the length of the first absorption layer 16.

In alternative aspects of the disclosure, opening 20 can have an extension in the longitudinal orientation between 10% and 100% of the length of the first absorption layer 16.

Opening 20 has a width perpendicular to the longitudinal direction of 15 mm but can vary in alternative aspects of the disclosure between 2 and 30 mm, preferably between 5 and 20 mm and even more preferably between 8 and 15 mm.

The second absorption layer 17 has longitudinal edges 32. A longitudinal compression 35 is disposed in the crotch area 26 of the second absorption layer 17 and coincides with the centre line 25. The compression 35 is clearly visible in comparison to the surrounding parts of the absorption layer 17. The thickness of the absorption layer 17 has been reduced by 20-90% in the central parts of the compression 35, preferably by 50-90% and even more preferably by 70-90%.

The compression 35 has its main extension in the longitudinal direction in the crotch area 26 but can also extend into both the front and the back end areas 27, 28.

The compression 35 extends in the longitudinal direction 30% of the length of the second absorption layer 17, which means that the compression 35 extends as long as the opening 20 in the first absorption layer 16.

In alternative aspects of the disclosure the compression 35 can have an extension in the longitudinal direction from 30% of the length of opening 20 in the first absorption layer 16 up to the entire length of the second absorption layer 17.

The compression 35 has a width perpendicular to the longitudinal direction of 15 mm but can have a width in alternative aspects of the disclosure between 2 and 30 mm, preferably between 5 and 20 mm and even more preferably between 8 and 15 mm.

The absorption body 15 is primarily distinguished in that the opening 20 disposed in the first absorption layer 16 and the compression 35 disposed in the second absorption layer 17 substantially overlap one another. Tests have shown that allowing the longitudinal opening in one layer and the longitudinal compression in the other layer to overlap one another shapes the article comprising the absorption body with substantially greater security in a desired manner when it is exposed to forces perpendicular to the longitudinal direction. Furthermore, an improved return force is obtained perpendicular to the longitudinal direction that effectively seals against the user's thighs, whereby many leakages are avoided.

The crotch area 26 of the absorption body 15 is shaped, when it is exposed to forces substantially perpendicular to the longitudinal centre line 25, substantially like a V with the second absorption layer 17, comprising compression 35, oriented toward the outside of the V shape and the first absorption layer 16, comprising opening 20, oriented inward into the V shape.

The compression 35 in the second absorption layer 17 functions, when the absorption body 15 is being formed, as a fold indication. The width of opening 20 in the first absorption layer 16 is reduced during the shaping and since the opening 20 does not contain any material that would be pressed together during the formation, the formation is not hindered either.

FIG. 2b shows how a special inlet material 45 divided into two parts is disposed on the side of the absorption body 15 that is intended to be turned facing the user during the use of an absorbent article comprising the absorption body 15.

The two parts of the inlet material 45 are substantially longitudinally extended and disposed in the crotch area 26 of the absorption body 15 but can also extend into the front or back part 27, 28 of the absorption body 15.

Each part of the two parts of the inlet material 45 is disposed transversely between the centre line 25 and the longitudinal edges 30 of the absorption body 15, wherein the outermost edges 46 of each respective transversal part of the inlet material 45 are disposed at an interval of 10-30 mm, preferably 15-30 mm from the longitudinal edges 30 of the absorption body 15.

Alternatively, the inlet material 45 is constituted by a material with a larger central opening, wherein the longitudinal outer edges of the inlet material 45 are disposed at the same distance from the longitudinal edges 30 of the absorption body 15 that are defined above for the two-part inlet material 45. It is also possible to conceive of an inlet material 45 without a central opening disposed with its edges at a distance from the longitudinal edges 30 of the absorption body.

The inlet material 45 comprises larger pores than the first absorption layer 16 against which the inlet material 45 rests. For example, the inlet material 45 consists of synthetic fibres of polyethylene, polypropylene or polyester. Also so-called two-component fibres comprising at least one of the fibre qualities polyethylene, polypropylene or polyester. The inlet material 45 has the task of receiving and temporarily storing liquid that strikes the article at a heavy flow rate in order to subsequently be drained off by the absorption body 15 at a slower rate.

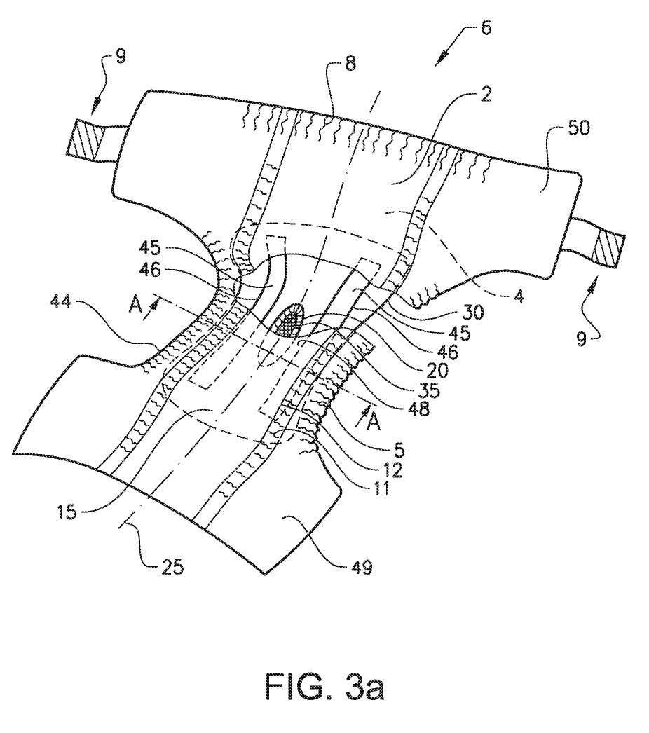

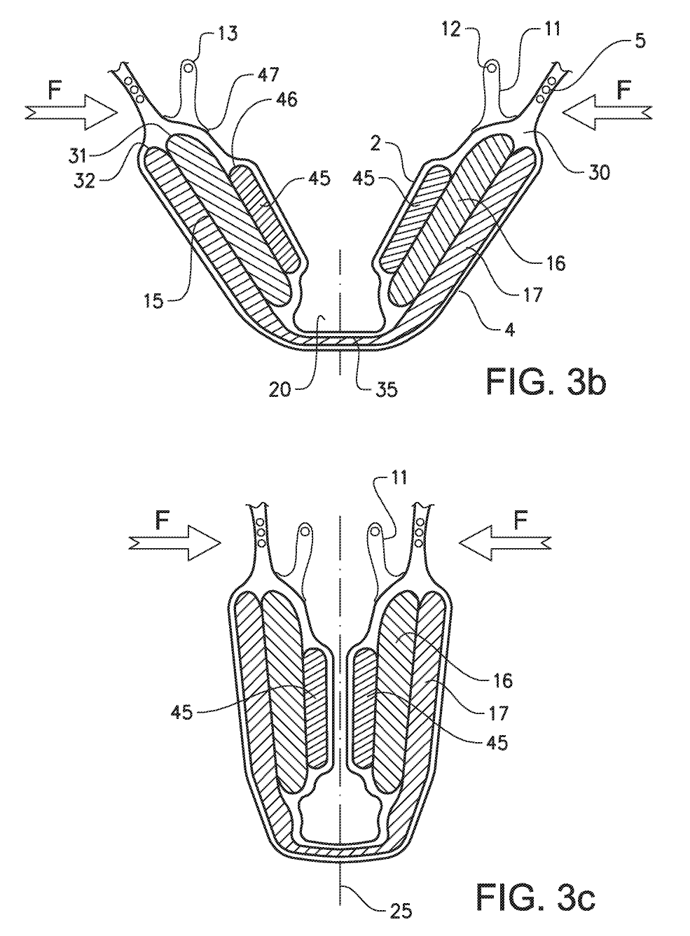

FIG. 3a shows an absorbent article 6 in conformity with a first aspect of the disclosure comprising an absorption body 15 in accordance with the above description.

Article 6 can be a baby diaper or an incontinence diaper intended for adult incontinent users.

The absorbent article 6 shown in FIG. 3a is a so-called all-in-one diaper but the cup shape specific for the disclosure is also relevant for alternative articles intended for urine absorption such as, for example, pant diapers and belt diapers.

The article 6 comprises a crotch area 48, a front end area 49 and a back end area 50. Furthermore, the absorbent article 6 comprises longitudinal edges 44.

The article 6 comprises a liquid-permeable casing layer 2 disposed over the surface of the article intended to be turned toward the user during use and comprises a backside layer 4 disposed over the surface of the article 6 and intended to be turned away from the user during use.

An absorption body 15 in conformity with the first aspect of the disclosure is disposed between the liquid-permeable casing layer 2 and the backside layer 4. The absorption body 15 is described in conjunction with the FIGS. 1, 2a and 2b above.

In order to better clarify how the article 6 is constructed under the liquid-permeable casing layer 2, an area of the latter has been opened up. The opened-up area shows, from the top to the bottom through the article 6, the two-part inlet material 45, the absorption body 15, the longitudinal opening 20 of the first absorption layer 16 and the longitudinal compression 35 of the second absorption layer 17. Furthermore, the opened-up area shows one of the longitudinal edges 30 of the absorption body 15.

The liquid-permeable casing layer 2 extends outside of the absorption body 15 along the entire circumference of the absorption body 15. The casing layer 2 can consist of any material suitable for the purpose. Examples of commonly occurring liquid-permeable casing materials are non-woven materials, perforated plastic films, plastic or textile nets and liquid-permeable foam layers.

In the areas between the edges 46 of the two-piece inlet material 45 arranged outermost in the transverse direction and the longitudinal edges 31, 32 of the absorption body 15, the liquid-permeable casing layer 2 has hydrophilic properties. The hydrophilic properties in these two edge areas mean that liquid that runs over the liquid-permeable casing layer 2 with increased likelihood will be trapped and transported into the absorption body 15. Hydrophilic properties can be obtained, for example, by treating the liquid-permeable casing layer 2 with surfactants.

Internal tests have shown that the hydrophilic properties of at least parts of the liquid-permeable casing layer 2 must have hydrophilic properties in accordance with the test method described below in order to ensure that urine is trapped and to avoid liquid from running up on the surface.

The backside layer 4 also extends outside the absorption body 15 along its entire circumference. Normally occurring backside layers are usually liquid-impermeable and consist of a thin, liquid-impermeable plastic film.

Backside layers 4 comprising a liquid-impermeable but vapour-permeable material are today in principle standard for many types of absorbent articles. The vapour permeability means that the article can breathe, which makes the article considerably more comfortable to use.

Backside layers comprising a laminate comprising a liquid-impermeable material functioning as liquid barrier and a textile-like material for making the article like an article of clothing on the outwardly oriented surface are also to be considered as more or less standard today.

The liquid-permeable casing layer 2 and the backside layer 4 are connected to one another outside the entire circumference of the absorption body 15.

The absorbent article 6 comprises elastic elements 5 constituting so-called leg elastic. The elastic elements 5 of the leg elastic extend in the longitudinal direction at least in the crotch area 48 of the absorbent article 6. The elastic elements 5 are transversely disposed between the respective longitudinal edge 30 of the absorption body 15 and the respective longitudinal edge 44 of the absorbent article 6. Furthermore, the elastic elements are disposed between the liquid-permeable casing layer 2 and the backside layer 4.

In alternative aspects of the disclosure the elastic elements 5 can be disposed on the side of the backside layer 4 that is intended to be turned away from the user during use.

The elastic elements 5 are applied in a tensioned state and are intended to raise the liquid-permeable casing layer 2 disposed outside of the absorption body 15 and backside layer 4 into a more upright configuration so that a sealing against the user's thighs is achieved, wherein liquid is prevented from running out over the edge of the absorbent article 6.

The leg elastic comprises three elastic elements 5 disposed between the liquid-permeable casing layer 2 and the backside layer 4 but can comprise several or fewer elastic elements in alternative articles.

In order to achieve the intended effect, that is, to seal against the user's thighs, the elastic elements 5 disposed along the one longitudinal edge 44 of the article 6 must have a return force between 0.30 N and 0.80 N at an extension of 70% of the accessible extension according to the test method described below.

Of course, the elastic elements 5 disposed along the opposite longitudinal edge 44 of the article 6 must also have the same return force.

In order to furthermore prevent liquid or faecal matter from leaking out over the edge of the absorbent article, article 6 is provided with inner side leakage barriers 11, so-called standing gathers. The inner side leakage barriers 11 are disposed in the vicinity of the longitudinal edges of the absorption body 15. The respective inner side leakage barriers 11 comprise, in connection with their free edge 13, an elastic element 12 connected to side leakage barriers 11 in the tensioned state (see FIG. 3b). The elastic elements 12 can alternatively be constituted by one or more elastic threads, elastic bands, elastic film, elastic foam or an alternative. Furthermore, the elastic elements 12 do not have to be disposed in connection with the free edge 13 of the inner side leakage barriers 11 but can be alternatively disposed between the free edge 13 and the attachment of the inner side leakage barrier 11 to the liquid-permeable casing layer 2 of the absorbent article 6.

The inner side leakage barriers 11 are constituted of separate, single folded material strips, wherein the shanks of the strips are connected to the liquid-permeable casing layer 2. Alternatively, the side leakage barriers 11 can be constituted of folds in the liquid-permeable casing layer 2.

When the tensioned elastic elements 12 are set free, they are contracted together with the inner free edges of the side leakage barrier 11, wherein the inner side leakage barriers 11 are brought to an upright configuration away from the liquid-permeable casing layer 2, at least in the crotch area 48 of the article 6.

In order to achieve an upright configuration of an inner side leakage barrier 11 it is required that the elastic elements 12 disposed in the barrier 11 have a return force between 0.20 N and 0.40 N at an extension of 70% of the accessible extension according to the test method described below.

The back end area 50 of the absorbent article 6 is provided with a so-called waist elastic 8 constituted by an elastic member disposed along the back transversal edge of the article 6. The waist elastic 8 is intended to give the absorbent article 6 a soft and flexible grip around the user's waist. In the described article only the back part is provided with waist elastic 8. In alternative articles the front part can also be provided with waist elastic.

Special fastening members 9 are arranged in the back end area 50 of the absorbent article 6 for connecting to the front end area 49 when the article is applied and disposed on a user.

FIG. 3b shows how the cross section A-A in FIG. 3a is shaped when the absorbent article 6 is exposed to lateral forces (F) from a user's thighs during use.

The absorption body 15 is disposed between the liquid-permeable casing layer 2 and the backside layer 4, as is described in conjunction with FIGS. 1 and 2.

The absorption body 15 is disposed with the first absorption layer 16 oriented toward the user and the second absorption layer 17 oriented away from the user during use.

The two-part inlet material 45 is disposed with its outermost edges 46 between the first absorption layer 16 and the liquid-permeable casing layer 2.

The inner side leakage barriers 11 are disposed adjacent the longitudinal edges 30 of the absorption body 15, wherein said inner side leakage barriers 11 comprise an inwardly oriented attachment 47 to the liquid-permeable casing layer 2.

The figure shows how the cup shape is formed in that the absorption body 15 folds around the longitudinal compression 35 of the second absorption layer 17, which compression constitutes an effective bending indication. The width of the opening 20 of the first absorption layer 16 is reduced at the same time in width when the absorption body 15 is bent/formed and due to the fact that the opening 20 does not contain any material that must be pressed together, no resistance is created during the bending/forming.

The cup shape allows that the urine which is not able to be absorbed by the absorption body 15 at the rate at which the urine is supplied can be temporarily stored in the cup shape formed.

As a result of the fact that the elastic elements 5 disposed between the liquid-permeable casing layer 2 of the article 6 and the backside layer 4 are applied in a tensioned state, said liquid-permeable casing layer 2 and backside layer 4 receive a substantially upright configuration and as a result of the fact that the elastic elements 12 of the inner side leakage barriers 11 are also applied in a tensioned state, the inner side leakage barriers 11 also receive an upright configuration during usage, as the figure shows.

FIG. 3c shows how the cross section A-A in FIG. 3a can be formed during use when the article is exposed to lateral forces at an angle perpendicular to the longitudinal direction from a user's thighs when the space between the user's thighs is small.

The crotch area of the article was compressed so much transversely thereby that the one part of the two-part inlet material 45 disposed on the one side of centre line 25 almost rests on the second part of the two-part inlet material 45 disposed on the opposite side of the centre line. In extreme cases it occurs that both parts of the two-part inlet material 45 lie on one another.

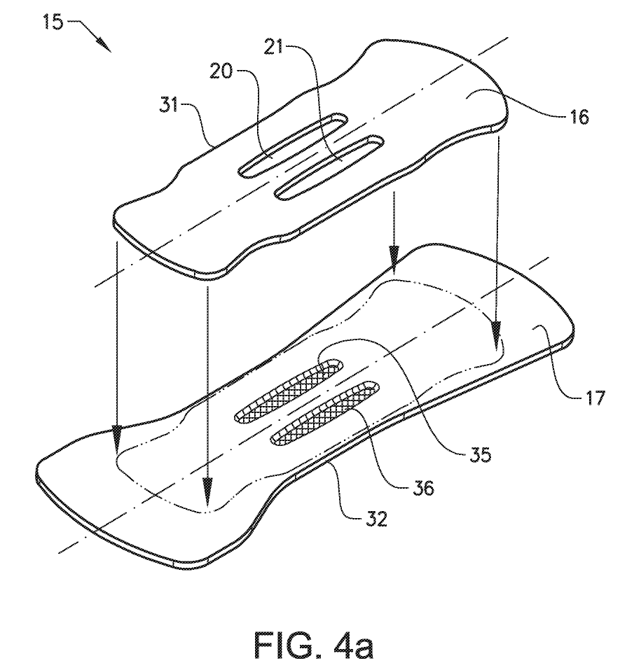

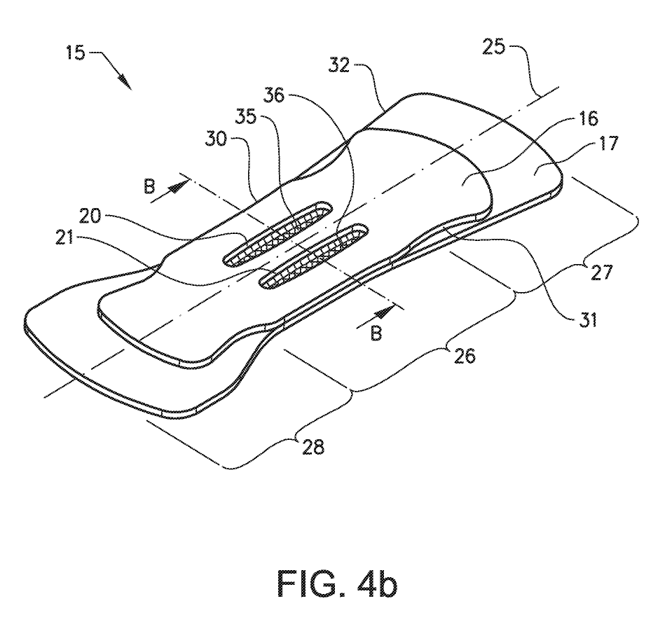

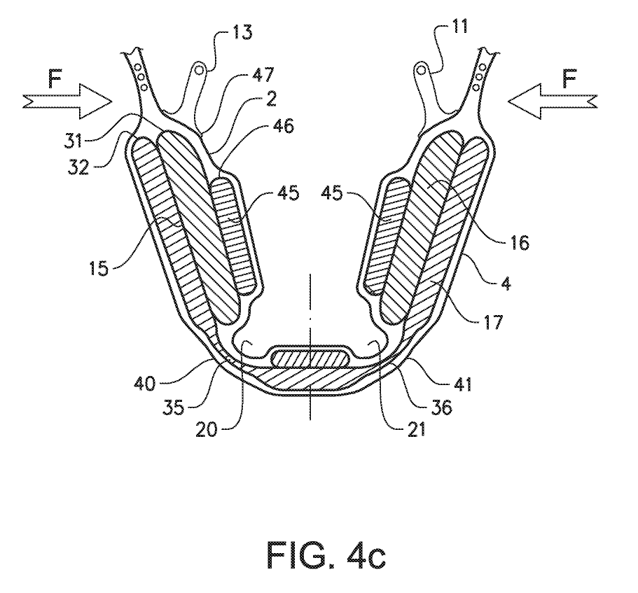

FIGS. 4a and 4b show an alternative absorption body 15 intended to be placed in an absorbent disposable article 6 in conformity with an alternative aspect of the disclosure. The absorption body 15 comprises a first absorption layer 16 and a second absorption layer 17 comprising at least cellulose fluff pulp.

In FIG. 4a the first and the second absorption layers 16, 17 of the absorption body 15 are separated from one another in order to increase the clarity while FIG. 4b shows the absorption body 15 such as it is disposed when it is applied into an absorbent article.

The absorption body 15 comprises a front end area 27, a back end area 28 and a crotch area 26 disposed between the end areas 27, 28 and comprises a longitudinal centre line 25.

The first absorption layer 16 has longitudinal edges 31.

Furthermore, the first absorption layer 16 comprises a first longitudinal opening 20 and a second longitudinal opening 21.

The openings 20, 21 have their main extensions in the longitudinal direction in the crotch area 26 but can also extend into both the front and the back end areas 27, 28.

The openings 20, 21 are disposed transversely on each side of the centre line 25 between said centre line 25 and the respective longitudinal edge 31.

The openings 20, 21 extend longitudinally over 30% of the length of the first absorption layer 16 but in alternative aspects of the disclosure they can have an extension longitudinally between 10% and 100% of the length of the first absorption layer 16.

The openings have a width perpendicular to the longitudinal direction of 15 mm but can vary in alternative aspects of the disclosure between 2 and 30 mm, preferably between 5 and 20 mm and even more preferably between 8 and 15 mm.

The second absorption layer 17 has longitudinal edges 32.

Furthermore, the second absorption layer 17 comprises a first longitudinal compression 35 and a second longitudinal compression 36.

The compressions 35, 36 have their main extensions in the longitudinal direction in the crotch area 26 but can also extend into both the front and the back end areas 27, 28. The compressions 35, 36 are disposed transversely on each side of the centre line 25 between said centre line 25 and respective longitudinal edge 32.

The compressions 35, 36 extend in the longitudinal direction 30% of the length of the second absorption layer 17, which means that the compressions 35, 36 extend as long as the openings 20, 21 in the first absorption layer 16.

In alternative aspects of the disclosure the compressions 35, 36 can have an extension in the longitudinal orientation from 30% of the length of openings 20, 21 in the first absorption layer 16 up to the entire length of the second absorption layer 17.

The compressions 35, 36 have a width perpendicular to the longitudinal direction of 15 mm but can have a width in alternative aspects of the disclosure between 2 and 30 mm, preferably between 5 and 20 mm and even more preferably between 8 and 15 mm.

The compressions 35, 36 are clearly visible in relation to the surrounding parts of absorption layer 17, wherein the thickness of the absorption layer 17 has been reduced by 20-90% in the central parts of the compression 17, preferably by 50-90% and even more preferably by 70-90%.

The absorption body 15 is primarily distinguished in that the first opening 20 disposed in the first absorption layer 16 substantially overlaps the first compression 35 disposed in the second absorption layer 17 and that the second opening 21 disposed in the first absorption layer 16 substantially overlaps the second compression 36 disposed in the second absorption layer 17.

The crotch area 26 of the absorption body 15 is shaped, when it is exposed to forces substantially perpendicular to the longitudinal centre line 25, like a U with the first absorption layer 16, comprising the openings 20, 21 oriented inward into the U-shape. The second absorption layer 17 comprising the compressions 35, 36 becomes oriented outward on the U shape.

The combination of overlapping opening—compression for the absorption body 15 according to this aspect of the disclosure functions in the same manner as was described above in connection with FIGS. 1 and 2, that is, the compressions function like bending indications that are not hindered by any material to compress in the openings.

FIG. 4c shows how the cross section B-B in FIG. 4b is formed when the absorption body 15 is disposed between a liquid-permeable casing layer 2 and a liquid-impermeable backside layer 4 in an absorbent article and when the article is exposed to lateral forces (F) from a user's thighs during use.

The absorbent article is preferably intended for the absorption of urine and can be, for example, a baby diaper or an incontinence article intended for adult users.

The absorption body 15 is disposed between the liquid-permeable casing layer 2 of the article and the backside layer 4, as is described in conjunction with FIGS. 4a and 4b above.

The absorption body 15 is disposed with the second absorption layer 17 oriented away from the user and the first absorption layer 16 oriented facing the user during use.

The longitudinal compressions 35, 36 of the second absorption layer 17 coil) operate with the longitudinal openings 20, 21 of the first absorption layer 16 and form two outwardly oriented side folds 40, 41 of the U shape.

The two-part inlet material 45 is disposed between the first absorption layer 16 and the liquid-permeable casing layer 2, and adjacent the longitudinal edges of the absorption body 15 are the inner side barriers 11 disposed.

The two outwardly oriented side folds 40, 41 are formed around the respective compression 35, 36 of the second absorption layer 17 which compressions constitute effective bending indications. Two longitudinal openings 20, 21 of the first absorption layer 16 are reduced in width when the side folds 40, 41 are bent/formed and due to the fact that the openings 20, 21 do not contain any material, no resistance is created during the bending/forming.

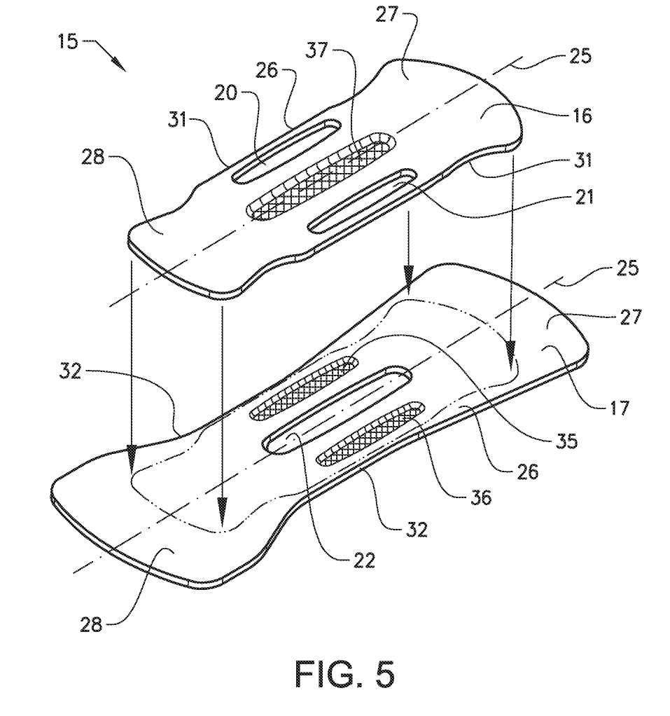

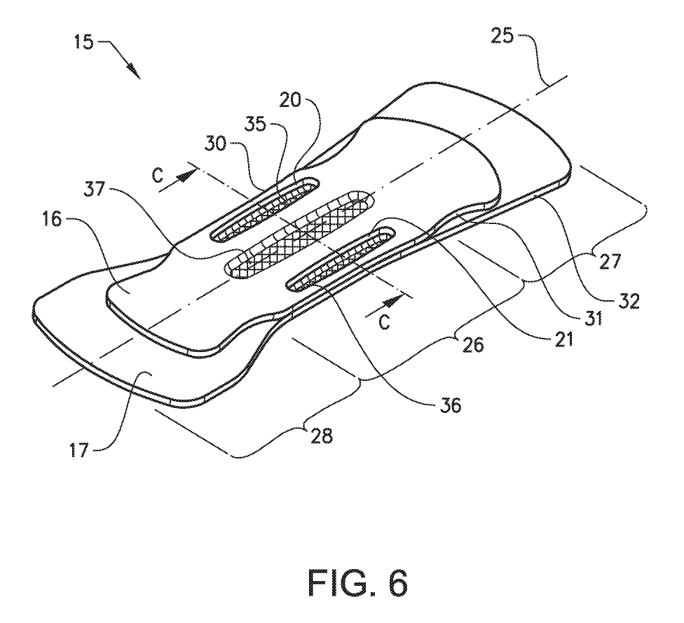

FIGS. 5 and 6 show an absorption body 15 provided to be placed in an absorbent disposable article 6 in conformity with a further aspect of the disclosure. The absorption body 15 is substantially constructed in the same way as the absorption body described in connection with the FIGS. 4a and 4b above but with the addition that the first absorption layer 16 comprises a third longitudinal compression 37 and that the second absorption layer 17 comprises a third longitudinal opening 22.

In FIG. 5 the first and the second absorption layers 16, 17 of the absorption body 15 are separated from one another in order to increase clarity while FIG. 6 shows the absorption body 15 such as it is disposed when it is applied into an absorbent article.

The third compression 37, coinciding with the centre line 25, has its main extension longitudinally in the crotch area 26 but can also extend into both the front and the back end areas 27, 28.

The compression 37 extends longitudinally 30% of the length of the first absorption layer 16 but in alternative aspects of the disclosure it can have a longitudinal extension from 30% of the length of opening 22 in the second absorption layer 17 up to the entire length of the first absorption layer 16.

Furthermore, compression 37 has a width perpendicular to the longitudinal direction of 15 mm but can have a width in alternative aspects of the disclosure between 2 and 30 mm, preferably between 5 and 20 mm and even more preferably between 8 and 15 mm.

The third longitudinal opening 22, coinciding with the centre line, has its main extension in the longitudinal direction in the crotch area 26 but can also extend into both the front and the back end areas 27, 28.

The opening 22 extends longitudinally over 30% of the length of the second absorption layer 17 but in alternative aspects of the disclosure it can have an extension longitudinally between 10% and 100% of the length of the second absorption layer 17.

The opening has a width perpendicular to the longitudinal direction of 15 mm but can vary in alternative aspects of the disclosure between 2 and 30 mm, preferably between 5 and 20 mm and even more preferably between 8 and 15 mm.

The absorption body 15 is primarily distinguished in that the third opening 22 disposed in the second absorption layer 17 and the third compression 37 disposed in the first absorption layer 16 substantially overlap one another and in that the first opening 20 disposed in the first absorption layer 16 substantially overlaps the first compression 35 disposed in the second absorption layer 17 and that the second opening 21 disposed in the first absorption layer 16 substantially overlaps the second compression 36 disposed in the second absorption layer 17.

The crotch area 26 of the absorption body 15 is formed, when it is exposed to forces substantially perpendicular to the longitudinal centre line 25, like a W with the first absorption layer 16, comprising the compression 37 and openings 20, 21 oriented inward into the W shape. The second absorption layer 17 comprising the opening 22 and the compressions 35, 36 becomes oriented outward on the W shape here.

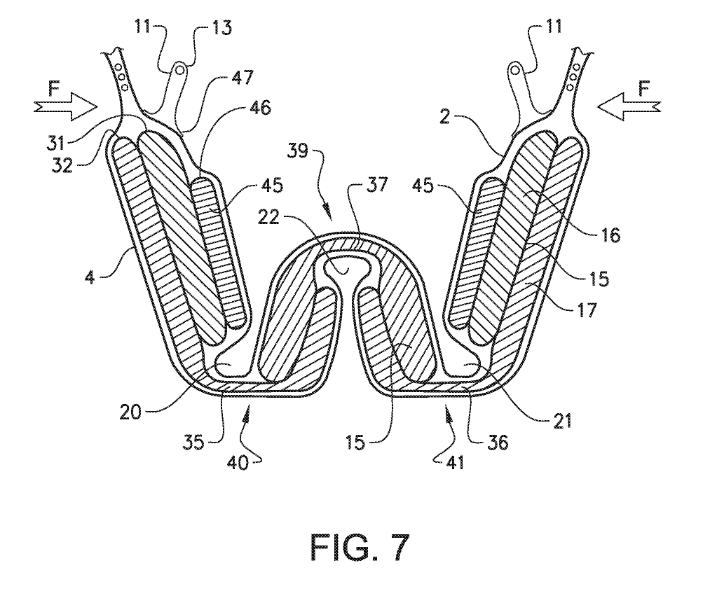

FIG. 7 shows how the cross section C-C in FIG. 6 is formed when the absorption body 15 is disposed between a liquid-permeable casing layer 2 and a liquid-impermeable backside layer 4 in an absorbent article and when the article is exposed to lateral forces (F) from a user's thighs during use.

The absorption body 15 is disposed between the liquid-permeable casing layer 2 and the backside layer 4 as is described in conjunction with FIGS. 5 and 6 above.

The absorption body 15 is disposed with the second absorption layer 17 oriented away from the user and the first absorption layer 16 oriented facing the user during use.

The longitudinal compressions 35, 36 of the second absorption layer 17 cooperate with the longitudinal openings 20, 21 of the first absorption layer 16 and form two outwardly oriented side folds 40, 41 of the W shape.

The two-part inlet material 45 is disposed between the first absorption layer 16 and the liquid-permeable casing layer 2 and adjacent the longitudinal edges of the absorption body 15 are the inner side leakage barriers 11 disposed.

The two outwardly oriented side folds 40, 41 are formed in the same manner as described above in conjunction with FIG. 4c.

The longitudinal third compression 37 of the first absorption layer 16 cooperates with the longitudinal opening 22 of the second absorption layer 17 and forms the inwardly oriented centre fold 39 of the W shape. The centre fold 39 is formed around the compression 37 of the first absorption layer 16, which compression constitutes an effective bending indication. The opening 22 of the second absorption layer is reduced in width when the centre fold 39 is bent/formed and due to the fact that the opening 22 does not contain any material, no resistance is created during the bending/forming.

The invention also comprises all conceivable combinations of the described embodiments of the invention.

Furthermore, the invention is not limited to the above-cited aspects of the invention but rather is naturally suitable for other combinations within the framework of the following claims.

### A Method for Determining the Return Force for the Elastic Elements of the Leg Openings and for the Elastic Elements of the Inner Side Leakage Barriers.

### DEFINITION

The return force P (X) is the elastic force for a sample when X % of the maximum accessible extension remains, for example, P (30) is the return force obtained when the sample is stretched out to 70% of the maximum accessible extension.

### Equipment

Absorbent articles
Tensile testing apparatus with a beam rate of 500 mm/min and 20 N load cell.
Equipment for being able to stretch the absorbent article out to be able to determine the accessible extension. Tape, paper clips, magnets or the like
Marking pen and scissors
Stopwatch
Ruler with mm scale
Flexible ruler with mm scale. Such flexible curve rulers can be purchased from scalerulers.com
Test protocol

### Preparation of the Sample for Determining the Return Force

A If the absorbent article 6 is a so-called pant diaper then the article is initially opened at the sides.
B In order to determine the maximum accessible extension of the elastic elements 5, 12 of the leg openings and/or of the inner side leakage barriers the absorbent article 6 is to be stretched out and its corners anchored on a flat support. The absorbent article is to be stretched out so that the material surrounding the elastic elements 5, 12 of the leg openings 11 and the inner side leakage barriers is substantially smoothed out, that is to say, substantially free of folds. The absorbent article 6 should not be stretched out so much so that the liquid-permeable casing layer 2 or the backside layer 4 is/are over-stretched but rather is to be only stretched out so that they are substantially smooth.

The anchoring of the corners of the absorbent article 6 in an extended state is suitably carried out with tape, paper clips, magnets or the like.

C Mark a distance of 200 mm±2 mm (100 mm±1 mm if the elastic elements of the article does not have an extended length that allows 200 mm) of the extended elastic element 5 of the leg openings and of the extended elastic element 12 of the inner side leakage barriers 11. The marked stretches should be substantially centred longitudinally around the middle points of the respective elastic elements 5, 12.

If the elastic elements 5 of the leg openings or the elastic elements 12 in the inner side leakage barriers 11 have a curved extension, 200 mm±2 is to be measured with the flexible ruler. The flexible ruler is to be formed according to the curved elastic elements 5, 11, after which 200 mm±2 is marked.

D If the intention is to determine the return force of the elastic elements 5 of the leg openings, lines parallel to the elastic elements 5 are to be marked at an interval of 5 mm±1 outside of and inside of the respective outermost and innermost disposed elastic elements 5 of the leg openings.

Strips are then cut out along the marked lines. The respective strips comprise all elastic elements 5 disposed at the respective leg opening.

The strips are to be cut as long as possible, preferably along the entire length of the absorbent article 6.

If it turns out that there is not 5 mm of material outside of the outermost disposed elastic elements 5 of the respective leg opening, no line shall be marked and no material shall be cut away either. A note about this is to be made in the test protocol.

If it turns out that the innermost disposed elastic elements 5 of the leg opening are disposed less than 5 mm from the longitudinal edge of the absorption body 15, no marking is to be made. In this case the cutting is to be made as close as possible to the longitudinal edge 30 of the absorption body 15. A note about this is to be made in the test protocol.

After the strips have been separated from the absorbent article 6 they are to remain entirely free of being extended for at least 30 minutes.

If the return force to be determined concerns the elastic elements 12 of the inner side leakage barriers 11, a first line is to be marked parallel with the elastic elements 12 at a distance of 5 mm±1 mm from the outermost disposed elastic element 12 in the direction of the free edge 13 of the respective inner side leakage barrier 11.

With the outermost disposed elastic element 12 is meant the elastic element 12 that is disposed closest to the free edge 13 of the respective inner side leakage barrier 11.

If it turns out that the distance between the outermost disposed elastic element 12 and the free edge 13 is less than 5 mm, no first line is to be marked. A note about this is to be made in the test protocol.

A second line is to be marked parallel with the elastic elements 12 at a distance of 5 mm±1 mm from the innermost disposed elastic element 12 in the direction of the connection of the inner side leakage barrier 11 with the liquid-permeable casing layer 2.

With the innermost disposed elastic element 12 is meant the elastic element 12 in the respective inner side leakage barrier 11 that is disposed the closest to the liquid-permeable casing layer 2.

If it turns out that the distance between the innermost disposed elastic element 12 and the liquid-permeable casing layer 2 is less than 5 mm, no second line is to be marked. A note about this is to be made in the test protocol.

Strips of the respective inner side leakage barrier 11 comprising all elastic elements 12 are now to be cut along the marked first and second lines.

If no first line according to the above is marked, no material is to be cut away and if no second line has been marked, the cutting is to be carried out as close as possible to the liquid-permeable casing layer 2.

The strips are to be cut as long as possible, preferably along the entire length of the absorbent article 6.

After the strips have been separated from the absorbent article 6 they are to remain entirely free of being extended for at least 30 minutes.

### Test

The cut strips are placed in the tensile test apparatus and stretched out to 70% of the accessible extension (200 mm±1 mm), that is, until the distance between the markings made is 140 mm (70 mm if the markings were made at a distance of 100 mm±1 mm).

70% of the accessible extension means that 30% of the accessible extension remains, from which the name P(30).

Register the sample's return force P(30) within 5 seconds after the completed extension.

In order to complete a test of the elastic elements 5 of the leg openings the elastic elements 5 are to be tested at both the right and the left leg opening in three absorbent articles 6.

In order for an absorbent article to pass the test, that is, be within the protective scope of the patent claims with respect to the return force of the elastic elements 5 of the leg openings, it is required that the average value of the 6 measurements defined above is in the interval of 0.30-0.80 N.

In order to complete a test of the elastic elements 12 disposed in the inner side leakage barriers 11, both the right and the left elastic elements 12 of the side leakage barrier 11 in three absorbent articles 6 are to be tested.

In order for an absorbent article to pass the test, that is, be within the protective scope of the patent claims, with respect to the return force of the elastic elements 12 of the inner side leakage barriers 11, it is required that the average value of the 6 measurements defined above is in the interval of 0.20-0.40 N.

1. An absorbent disposable article comprising a liquid-permeable casing layer intended to be facing the user and a substantially liquid-impermeable backside layer facing away from the user during use and an absorption body comprising longitudinal edges disposed between the liquid-permeable casing layer and the substantially liquid-impermeable backside layer, wherein the article comprises a longitudinal centre line, a crotch area a front end area and a back end area, wherein the absorption body comprises a first absorption layer facing the user during use and a second absorption layer facing from the user during use, wherein the liquid-permeable casing layer and the backside layer extend outside of the longitudinal edges of the absorption body, wherein at least one elastic element is disposed between the liquid-permeable casing layer and the backside layer outside of the respective longitudinal edge of the absorption body, wherein the elastic elements have a return force between 0.30 N and 0.80 N at an extension of 70% of the accessible extension according to the test method described herein, wherein the first absorption layer comprises a first longitudinal opening disposed in the crotch area and wherein the second absorption layer comprises a first longitudinal compression disposed in the crotch area, wherein the first longitudinal opening and the first longitudinal compression overlap one another.

2. An absorbent disposable article according to claim 1, wherein the first longitudinal opening of the absorption body and the first longitudinal compression substantially coincide with the longitudinal centre line.

3. An absorbent disposable article according to claim 1, wherein the first absorption layer of the absorption body comprises a second longitudinal opening disposed in the crotch area between the longitudinal centre line and one one longitudinal edge of the absorption layer, and wherein the first longitudinal opening is disposed between the longitudinal centre line and the opposite longitudinal edge of the absorption layer, wherein the second absorption layer comprises a second longitudinal compression disposed in the crotch area between the longitudinal centre line and one longitudinal edge of the absorption layer, and wherein the first longitudinal compression is disposed between the longitudinal centre line and the opposite longitudinal edge of the absorption layer, wherein the second longitudinal opening and the second longitudinal compression overlap one another.

4. An absorbent disposable article according to claim 3, wherein the first absorption layer comprises a third longitudinal compression disposed in the crotch area substantially coinciding with the longitudinal centre line, and wherein the second absorption layer comprises a third longitudinal opening disposed in the crotch area substantially coinciding with the longitudinal centre line, wherein the third longitudinal compression and the third longitudinal opening overlap one another.

5. An absorbent disposable article according to claim 1, wherein the article comprises at least one inner side leakage barrier disposed on each side of the longitudinal centre line of the article, wherein the respective inner side leakage barrier is disposed in the vicinity of the respective longitudinal edge of the absorption body at least in the crotch area, wherein the respective inner side leakage barrier comprises a free edge, wherein the respective inner side leakage barrier comprises at least one tensioned elastic element.

6. An absorbent disposable article according to claim 5, wherein the elastic elements disposed in the respective inner side leakage barrier have a return force between 0.20 N and 0.40 N at an extension of 70% of the accessible extension according to the described test method.

7. An absorbent disposable article according to claim 1, wherein an inlet material is disposed on the side of the first absorption layer that is intended to be arranged toward the user.

8. An absorbent disposable article according to claim 7, wherein the inlet material is divided into 2 separate parts, wherein one part is disposed between the longitudinal centre line and one longitudinal edge of the absorption body and the other part is disposed between the longitudinal centre line and the other longitudinal edge of the absorption body, wherein the respective part has a distance to the longitudinal centre line and to the adjacent longitudinal edge.

9. An absorbent disposable article according to claim 1, wherein the first absorption layer and the second absorption layer of the article have substantially the same surface weights.

10. An absorbent disposable article according to claim 1, wherein the respective absorption layers of the article have a surface weight between 50 g/m2 and 1000 g/m2.

11. An absorbent disposable article according to claim 1, wherein the respective absorption layers of the article have a content of highly absorbent polymer material between 0% and 80%.

12. An absorbent disposable article according to claim 1, wherein one of the absorption layers of the article has a greater longitudinal extension than the other absorption layer.

13. An absorbent disposable article according to claim 1, wherein the front end area and the back end area of the absorption body have a greater transversal width than the crotch area.

14. An absorbent disposable article according to claim 1, wherein the disposable article is a baby diaper.

15. An absorbent disposable article according to claim 1, wherein the disposable article is an incontinence diaper intended for adult incontinent users.
